(19) United States
(12) Patent Application Publication
(10) Pub. No.: US 2009/0253801 A1
(43) Pub. Date: Oct. 8, 2009

(54) Novel Antiarrhythmic Drug Formulations

(76) Inventors: Manish J. Shah, West Caldwell, NJ (US); Ray J. Difalco, Ridgewood, NJ (US)

Correspondence Address: Clements Bernard PLLC, 1901 Roxborough Road, Suite 250, Charlotte, NC, 28211, US

(21) Appl. No.: 12/419,790

(22) Filed: Apr. 7, 2009

Related U.S. Application Data
(60) Provisional application No. 61/042,900, filed on Apr. 7, 2008.

(51) Int. Cl. A61K 31/138; A61P 9/00

(52) U.S. Cl. 514/652

(57) ABSTRACT

The present invention provides a method of achieving a novel Propafenone formulation, which will have reduced number dosings and improved bioavailability. The present invention also applies to the salt and active metabolites of the parent and pro-drug. The novel compositions are designed to release Propafenone after oral intake in a manner, which enables absorption to take place in the gastrointestinal tract so that a relatively fast peak plasma concentration of the active is obtained. The time-specific pharmaceutical formulation, the disintegration of which is triggered by time lapse and the pH of the environment to which it is subjected. The novel compositions are also designed for administration once or twice daily. i.e. a therapeutically effective concentration of Propafenone is maintained for a period of at least 10-20 hours. A composition is designed to release Propafenone in at least the following consecutive steps: i) an initial relatively fast release of Prop afenone, ii) a second rise in release of Propafenone about 1-6 hours after oral intake; and iii) a third rise in release of Propafenone about 4-15 hours after oral intake, depending upon ones metabolism.

### CROSS-REFERENCE TO RELATED APPLICATION(S)

The current application claims the benefit of the earlier priority filing date of the provisional application Ser. No. 61/042,900, that was filed on Apr. 7, 2008.

### BACKGROUND OF THE INVENTION

1. Field of the Invention

This invention is concerned with a delivery system for a drug that is metabolized by the liver of a human or animal that has a portion that immediately release the drug and another portion that has a delayed release, but upon being released is quick and similar to the first portion (but it could be somewhat slower than the first portion). For example, an antiarrhythmic drug, such as a beta blocker pharmaceutical composition used in the treatment of cardiac arrhythmia is metabolized quickly by the liver. The present invention allows the drug to be taken once a day, for example and with a lesser amount than taken multiple times a day.

2. Prior Art

Cardiac arrhythmia is a disorder involving the electrical impulse generating system of the heart. The antiarrhythmic action of Propafenone appears to be due to its propensity to depress conduction and prolong the refractory period in myocardium. Propafenone is known to undergo extensive and saturable presystemic biotransformation which results in dose and dosage form dependent absolute bioavailability; e.g. 150 mg immediate release tablets had an absolute bioavailability of 3.4%, while 300 mg immediate-release tablet had an absolute bioavailability of 10.6%. Absorption from 300 mg solution was rapid, with an absolute bioavailability of 21.4%.

However, from person to person there are marked genetically determined differences in the rate of hepatic Propafenone metabolism. About 10% of the U.S. Caucasian population is considered “poor metabolizes” and 90% is considered “extensive metabolizes”. Consequently, many individuals metabolize current formulations of Propafenone too quickly. This results in more frequent administration of the dosage form or higher daily dose of Propafenone to obtain the similar exposure to Propafenone.

In “extensive metabolizes”, the bioavailability of Propafenone from Rythomol SR (extended release) twice a day formulation was less than that of the immediate release formulation as the more gradual release of Propafenone from the prolonged-release preparation resulted in an increase in overall first pass metabolism. As a result of the increased first pass effect, higher daily doses of Propafenone were required from the slow release formulation relative to the immediate release formulation to obtain the similar exposure to Propafenone. For instance one SR 325-mg capsule administered twice a day has similar bioavailability to one immediate release 150-mg tablets administered three times a day. As a result more Propafenone from the slow release capsule formulation undergoes hepatic metabolism into 5-hydroxy and hydroxy-methoxy metabolites.

Thus with a drug that metabolizes in the liver, like Propafenone HCL, slow release or sustained release only permits the liver to metabolize the drug such that the bioavailability is low—to little drug made its way into the blood stream and thus it is pharmaceutically insufficient. Thus there is a need for a delivery system that can delay the release of at least a portion of a drug that metabolizes in the liver, but when it is released, it must be released immediately so that the liver does not have time to metabolize all the drug and an effective amount of the drug enters the blood stream, i.e. have a high bioavailability.

### SUMMARY OF THE INVENTION

It is therefore an object of the present invention to provide a method of preparing a pharmaceutical formulation, which releases the pharmaceutical active drug faster than the slow release capsule formulation, and also provide delay in the drug release from the formulation, which is pH and/or time specific extended-release to allow once-a-day dosing. More specifically, the chief aim of the present invention is to first release some of the drug immediately, while delay releasing another portion of the drug, but when it is released, it is quick and similar to the first portion (but it could be somewhat slower than the first portion).

The type of drugs that can beneficially employ this type of delivery system are drugs that are metabolized by the liver, and in particular (but not limited to) drugs known as beta blockers, typically useful for cardiac arrhythmias and cardio protection after myocardial infarction and for hypertension. Beta blockers may also be referred to as beta-adrenergic blocking agents, beta-adrenergic antagonists, or beta antagonists. Typical beta blockers are propafenone, oxprenolol, pindolol, labetalol, and carvedilol. This list is not meant to be exhaustive and those skilled in the art certainly know those drugs that are metabolized by the liver.

As a first aspect, the present invention provides a method of improving bioavailability thereby reducing the dose of a pharmaceutically active agent to a patient in need of the therapeutic effects of pharmaceutically active agent. The method includes administrating a pharmaceutical formulation comprising a rapid releasing core containing the pharmaceutically active agent to be delivered.

As a second aspect, the present invention provides a method of decreasing the frequency of administration from three times a day to once-a day or twice-a day. The method includes producing three different releasing cores by applying three different coatings onto the rapid-releasing core. These cores then release at a different time but the release rate is very similar to the rapid releasing core.

The foregoing and other objects and aspects of the present invention are explained in detail in the detailed description and examples set forth herein below.

### DETAILED DESCRIPTION OF THE INVENTION

The present invention relates, for example, to drugs used to treat cardiac arrhythmias include:

Class I—Sodium Channel Blockers

Disopyramide (Norpace)

Flecainide (Tambocor)

Lidocaine (Xylocaine)

Mexiletine (Mexitel)

Moricizine (Ethmozine)

Procainamide (Procan, Procanabid, Pronestyl)

Propafenone (Rythmol)

Quinidine (Various)

Tocainide (Tonocard)

Class II—Beta Blockers

Acebutolol (Sectral)

Atenolol (Tenormin)

Betaxolol (Kerlone)

Bisoprolol (Zebeta)

Carvedilol (Coreg)

Esmolol

Metoprolol (Toprol, Lopressor)

Nadolol (Corgard)

Propranolol (Inderal)

Sotolol (Betapace)

Timolol (Blocadren)

Class III—Potassium Channel Blockers

Amiodarone (Cordarone, Pacerone)

Azimilide (Stedicor)

Bepridil

Dofetilide (Tikosyn)

Ibutalide (Corvert)

Sotalol (Betapace)

Tedisamil

Class IV—Calcium Channel Blockers

Diltiazem (Cardizem, Tiazac)

Verapamil (Calan, Covera, Isoptin)

Miscellaneous

Adenosine (Adenocard)

Digoxin (Lanoxin)

Ibutilide

Trecitilide

Ersentilide

Dofetilide

Dronedarone

Azimilide

Tedisamil (drugs that are both Ca2+ channel and beta-blockers).

Although this invention is described with respect to Propafenone HCL, the invention is not limited thereto. Propafenone HCL (usually called Propafenone) is an antiarrhythrnic drug and is metabolized by the liver. Propafenone is an antiarrhythmic drug available for oral and intravenous administration. The chemical name for PrTpafenone is: 2′-[3-(propylamino)-2-(hydroxy)-(propoxy)-3-phenylpropiophenone hydrochloride, also known as an IUPAC name of 1-[2-(2-hydroxy-3-propylamino-propoxy)phenyl]-3-phenyl-propan-1-one. Molecular formula: C₂]H₂₇NO₃—HCl. Molecular weight: 377.92. Propafenone occurs as colorless crystals or white crystalline powder with a very bitter taste. It is slightly soluble in water, chloroform and ethanol.

In principle any relevant controlled formulation techniques for preparing an oral controlled release composition may be applied. Examples of different controlled release teclnologies are:

1. Single Units:

coated matrix,

double or triple compression and

multilayer coating

2. Multiple Units:

units having controlled release compression coating

units having controlled release matrix

units having controlled release compression coating and

units with multilayer coating

In specific embodiments, a composition according to the invention is in the form of a solid dosage form such as, e.g., tablets capsules, granules, pellets, solid dispersion and the like.

Also covered in this invention is the different commercially available grade (particle size & bulk density) of Propafenone hydrochloride. Depending upon the formulation examples presented in this invention, micronized grade, fme grade, and coarse grade were used.

Apart from the active drug substance in the composition, a pharmaceutical composition according to the invention may fiurther comprise pharmaceutically acceptable excipients. A pharmaceutical acceptable excipient may be added to the active drug substance with the purpose of making it possible to obtain a pharmaceutical composition, which has acceptable technical properties.

Examples of coating material which can be used as a matrix-forming agents, are polymers and film-forming agents covered in the present invention as a component (a) ethyl cellulose, a quaternary anmmonium acrylic or methacrylic polymers, an acrylic or a methacrylic ester copolymers or a mixture thereof; and as component (b), cellulose acetate phthalate, hyoxypropyl methylcellulose phthalate, polyvinyl acetate phthalate, methacrylic acid:acrylic ester copolymer, hydroxypropyl methlycellulose acetate succinate, shellac, cellulose acetate trimellitate, and mixture thereof; and as component (c), polyvinylpyrrolidone, hydroxypropyl cellulose, hydroxypropyl methylcellulose, polyethylene glycol having a molecular weight of from 1700 to 20,000 polyvinyl alcohol and monomers therefor and mixtures thereof. Common tradenames include, various grades of Eudragits (all from Rohm), Sureteric (from Colorcon), Aquateric (from FMC) or HPMCP (from Shin-Etsu).

The amount of coating applied is adapted so as to obtain a predetermined dissolution characteristic of the composition. However, the amount of coating applied should also be adapted so that there will be no rupturing problems.

The coating may be admixed with various excipients such as plasticizers, anti-adhesive such as, e.g., colloidal silicone dioxide, inert fillers, lipophillic agents such as, e.g., amylose, ethylcellulose, Eudragits S 12.5 etc., and pigments used in a manner known per se. Tackiness of the water-dispersible film forming substance may be overcome by simply incorporating an anti-adhesive in the coating. Examples of anti-adhesive are metallic stearates, microcrystalline cellulose, calcium phosphate, and talc.

Examples of plasticizers for use in accordance with the present invention include triacetin, acetylated monoglyceride, olive oil, acetyl tributyl citrate, acetyl triethyl citrate, glycerin, sorbitol, polyethylene glycol, and polypropyleneglycol.

Fillers/diluents/binders may be incorporated such as sucrose, sorbitol, mannitol, various grades of lactose, various grades of microcrystalline cellulose, dextrins, maltodextrins, starches or modified starches, sodium phosphate, calcium phosphate, calcium carbonate, gelatin, polyvinylpyrrilidone, and sodium carboxymethlycellulose.

Disintegrants may be used such as cellulose derivatives, including microcrystalline cellulose, low-substituted hydroxypropyl cellulose, croscarmellose sodium, alginic acid, insoluble polyvinlypyrrolidone, and sodium carboxymethyl starch.

Glidents and lubricants may be incorporated such as stearic acid, metallic stearates, talc, waxes, and glycerides with high melting temperatures, colloidal silica, sodium stearyl frrmarate, polyethyleneglycols, and alkyl sulphates.

Surfactants may be employed such as non-ionic (various grades of polysorbate); anionic such as docusate sodium and sodium lauryl sulfate, and cationic such as (benzalkonium chloride. Examples of amphoteric surfactant is 1,2-diacyl-L-phosphatidylcholine.

Other appropriate pharmaceutically acceptable excipients may include colorants, flavoring agents, pH adjusting agents, solubilizing agents, wetting agents and buffering agents.

In the preferred embodiments for coating material, the acrylic coating is an acrylic lacquer used in the form of an aqueous dispersion, that is commercially available from Rohrn Pharma under the tradename Eudragit RTM. In further preferred embodiments, the acrylic coating comprises a mixture of two acrylic lacquers.

The preferred binder is hydroxyethyl methylcellulose, filler is microcrystalline, plasticizer is triethyl citrate, antiadhesive agent is talc, disintegrant is Ac-Di-Sol. The surfactants used to enhance the drug release were sodium lauryl sulfate & Tween 80. The lubricant used is magnesium stearate.

A preferred composition of the coated propafenone according to present invention is a multiple unit dosage form such as, e.g., wherein at least one of the at least two parts comprises at least two different types of pellets, the first type of pellet corresponding to a first fraction and the second type of pellet corresponding to a second fraction.

A composition according to the invention may also include individual units containing tablets, i.e., be in the form of multiple unit dosage form comprising at least two different types of tablets, the first type of tablet corresponding to the first part, and second type of tablet corresponding to the second part.

Another composition according to the invention may be in the form of a multiple unit dosage form, wherein the first or the second part, or all of the parts, is in the form of tablets, in the form of mini-tablets, in the form of pellets, in the form of granules or in the form of large crystals of the active substance.

Another aspect of the composition according to present invention may have first part, a second part, a third part and/or fourth part which has release kinetics very similar to each other (relatively fast) but at a different time and under different pH environment.

Dissolution profile as used herein, means a plot of active ingredient released as a finction of time. A profile is characterized by the test conditions selected. Thus the dissolution profile may be generated at a preselected shaft speed, temperature, and pH of the dissolution media. A first dissolution profile may be measured at a pH level approximating that of the stomach. At least a second dissolution profile may be measured at pH levels approximating that of least one point in the intestine. A highly acidic pH (1 to 5) may simulate the stomach and a less acidic to basic pH (greater then 5 to approximately 7.5) may simulate the intestine.

Accordingly in a further preferred aspect, a first dissolution is measured at a pH level approximating that of the stomach and a second dissolution profile is measured at a pH level approximating that of at least on point in the intestine; the first and second dissolution profiles for the novel release composition each being equal to or greater than the minimum dissolution required to provide substantially equivalent bioavailability in the form of a capsule, tablet or liquid containing at least one active ingredient in an immediate release form. Moreover, since the coating is insoluble at an acidic pH, for example as encountered in the stomach of the patients, the dissolution profile of these coated parts will only be tested in specific pH in which the coating dissolves.

The following examples are intended to illustrate specific embodiments of the present invention but are not intended in any way to limit the invention, and especially are not intended to limit the invention to just Propafenone. Propafenone is described herein for purposes of clearly illustrating the invention, but not limiting it. Some of the examples are included in order to illustrate that varying a number of formulation parameters can change the release rate and dissolution characteristics of the composition.

### EXAMPLE 1

Composition Made in the Form of Capsules Containing Multiple Units.

The Propafenone controlled release product is prepared by manufacturing one type of tablet, which afterwards is coated with different types of film coating material. The tablets are used to create a capsule. The capsule ends up with 4 different types of tablets (one non-coated tablet, one EC-coated tablet, one delayed EC-coated tablet, and a prolonged EC-coated tablet). EC means “enteric coated”.

Core Tablet Preparation:

Propafenone hydrochloride 150 mg

Croscannellose sodium 10 mg

Methocel E6 LV powder 3.8 mg

Purified water 34.2 mg (Methocel is dissolved in Purified water)

Additional purified water—15 mg

Magnesium Stearate 1.2 mg

The active drug Propafenone and Croscarmellose sodium is wetted with the Methocel/purified water solution and granulation is performed at an appropriate time & mixing intensity. The additional purified water may be added to achieve the desired granulation. The drying of the wet granulate is carried out in a Glatt fluid bed The granules are dried to LOD (loss on drying) of not more than 2 wt. %. WHAT IS LOD—level of demand? The dried granulate is passed through a suitable sieve (#20 mesh). Magnesium Stearate is sieved through a #40 mesh and mixed with the sized granules in a V-Blender for 5 minutes. The tablet is compressed at 165 mg weight and hardness of 3-7 kp.

Step 1—Non Coated Tablets

One batch of above Core tablets is not coated because it is used as an immediate-release unit. The tablet is a part of the content in the capsule.

Step 2—EC Coated Tablet

One batch of Core tablets is coated with an outer coat in a conventional perforated coating pan with a spray nozzle 1.5-mm and a spray pressure of 2.5 bar. The composition for the coating is shown below:

Pan Load 500 g

Eudragit L30 D 55 dispersion 260 g

Triethyl Citrate 8 g

Talc 20 g

Purified water 183 g

10 weight % of dry matter was applied, calculated in the percentage of the core weight. It is possible to easily vary the coating level by adding certain pharmaceutical excipients from 1 to 20 wt. %. Throughout the coating process the bed temperature was maintained in the interval from 30-38° C. by adjustment of the liquid inflow rate of the inlet temperature. The inlet air temperature was kept at approximately 45-55° C.

Step 3—Delayed EC Coated Tablet

One batch of Core tablets is coated with an outer coat in a conventional perforated coating pan with a spray nozzle 1.5-mm and a spray pressure of 2.5 bar. The composition for the coating is shown below:

Pan Load 500 g

Eudragit L30 D 55 dispersion 130 g

Eudragit FS 30 D dispersion 130 g

Triethyl Citrate—8 g

Talc—20 g

Purified water—183 g

10 weight % of dry matter was applied, calculated in the percentage of the core weight. It is possible to easily vary the coating level by adding certain pharmaceutical excipients from 1 to 20 wt. %. Throughout the coating process the bed temperature was maintained in the interval from 30-38° C. by adjustment of the liquid inflow rate of the inlet temperature. The inlet air temperature was kept at approximately 45-55° C.

Step 4—Prolonged EC Coated Tablet

One batch of core tablets is coated with an outer coat in a conventional perforated coating pan with a spray nozzle 1.5-mm and a spray pressure of 2.5 bar. The composition for the coating is shown below:

Pan Load 500 g

Eudragit FS 30 D dispersion 260 g

Triethyl Citrate—4 g

Talc—39 g

Purified water—234 g

10 weight % of dry matter was applied, calculated in the percentage of the core weight. It is possible to easily vary the coating level by adding certain pharmaceutical excipients from 1 to 20 wt. %. Throughout the coating process the bed temperature was maintained in the interval from 30-38° C. by adjustment of the liquid inflow rate of the inlet temperature. The inlet air temperature was kept at approximately 45-55° C.

Capsule Filling

The 4 different tablets (steps 1,2 3 & 4) were filled into capsules by hand. The amount is shown below:

1 unit Non-coated tablet containing 150 mg Propafenone HCl

1 unit EC-coated tablet containing 150 mg Propafenone HCl

1 unit delayed EC-coated tablet containing 150 mg Propafenone HCl

1 unit prolonged EC-coated tablet containing 150 mg Propafenone HCl

### EXAMPLE 2

Composition Made in the Form of Capsules Containing Multiple Units.

The Propafenone controlled release product is prepared by manufacturing four types of tablets. The capsule ends up with 4 different types of tablets (one non-coated tablet, one EC-coated tablet, one delayed EC-coated tablets and prolonged EC-coated tablets). The different between Example 1 & 2 is the method of producing 4 types of tablets.

Step 1—Core Tablet Preparation:

These tablets do not contain any polymrer coating because it is used as an immediate-release unit. The tablet is a part of the content in the capsule.

Propafenone hydrochloride 150 mg

Croscarmellose sodium 10 mg

Methocel E6 LV powder 3.8 mg

Purified water 34.2 mg (Methocel is dissolved in Purified water)

Additional purified water—15 mg

Magnesium Stearate 1.2 mg

The active drug Propafenone and Croscarmellose sodium is wetted with a Methocel/purified water solution and granulation is performed at an appropriate time & mixing intensity. The granulation can be performed in a conventional planetary or high shear mixer and fluid bed dryer using the top spray technique. The additional purified water may be added to achieve the desired granulation. The drying of the wet granulate is carried out in a Glatt fluid bed. The granules are dried to LOD of not more than 2 wt. %. The dried granulate is passed through a suitable sieve (#20 mesh). Magnesium Stearate is sieved through a #40 mesh and mixed with the sized granules in a V-Blender for 5 minutes. The tablet is compressed at 165-mg weight and hardness of 3-7 kp.

Step 2—Matrix Tablet

The composition of matrix tablets is shown below:

Propafenone hydrochloride 150 mg

Croscarmellose sodium 10 mg

Methocel E6 LV powder 1.9 mg

Purified water 34.2 mg

Eudragit L30D dispersion 38 mg

Additional Purified water 5 mg

Avicel PH 102 15 mg

Magnesium Stearate 1.2 mg

The active drug Propafenone and Croscarmellose sodium is wetted with a Methocel/purified water solution and Eudragit L30D dispersion and granulation is performed at an appropriate time & mixing intensity. The granulation can be performed in a conventional planetary or high shear mixer and Fluid bed dryer/coater using Top spray technique. The additional purified water may be added to achieve the desired granulation. The drying of the wet granulate is carried out in a Glatt fluid bed. The granules are dried to LOD of not more than 2 wt. %. The dried granulate is passed through a suitable sieve (#20 mesh). Avicel PH 102 and Magnesium Stearate is sieved through a #40 mesh and mixed with the sized granules in a V-Blender for 5 minutes. The tablet is compressed at 189.5-mg weight and hardness of 3-7 kp. The amount of Eudragit used in the process can be easily varied more 50 wt. % on either side by adding/deleting generally acceptable pharmaceutical ingredients described earlier.

Step 3—Delayed Matrix Tablet

The composition of matrix tablets is shown below:

Propafenone hydrochloride 150 mg

Croscarmellose sodium 10 mg

Methocel E6 LV powder 1.9 mg

Purified water 34.2 mg

Eudragit NE 30D dispersion 19 mg

Eudragit FS30 D dispersion 19 mg

Additional Purified water 5 mg

Avicel PH 102 15 mg

Magnesium Stearate 1.2 mg

The active drug Propafenone and Croscarmellose sodium is wetted with an Eudragit N30D & FS30D dispersion and granulation is performed at an appropriate time & mixing intensity. The granulation can be performed in a conventional planetary or high shear mixer and fluid bed dryer using a top spray technique. The additional purified water may be added to achieve the desired granulation. The drying of the wet granulate is carried out in a Glatt fluid bed. The granules are dried to LOD of not more than 2 wt. %. The dried granulate is passed through a suitable sieve (#20 mesh). Avicel PH 102 and Magnesium Stearate is sieved through a #40 mesh and mixed with the sized granules in a V-Blender for 5 minutes. The tablet is compressed at 189.5-mg weight and hardness of 3-7 kp. The amount of Eudragit used in the process can be easily varied more 50 wt. % on either side by adding/deleting generally acceptable pharmaceutical ingredients described earlier.

Step 4—Prolonged Matrix Tablet

The composition of matrix tablets is shown below:

Propafenone hydrochloride 150 mg

Croscarmellose sodium 10 mg

Methocel E6 LV powder 1.9 mg

Purified water 34.2 mg

Eudragit FS30 D dispersion 38 mg

Additional Purified water 5 mg

Avicel PH 102 15 mg

Magnesium Stearate 1.2 mg

The active drug Propafenone and Croscarmellose sodium is wetted with an Eudragit N30D & FS30D dispersion and granulation is performed at an appropriate time & mixing intensity. The granulation can be performed in a conventional planetary or high shear mixer and fluid bed dryer using a top spray technique. The additional purified water may be added to achieve the desired granulation. The drying of the wet granulate is carried out in a Glatt fluid bed. The granules are dried to LOD of not more than 2 wt. %. The dried granulate is passed through a suitable sieve (#20 mesh). Avicel PH 102 and Magnesium Stearate is sieved through a #40 mesh and mixed with the sized granules in a V-Blender for 5 minutes. The tablet is compressed at 189.5-mg weight and hardness of 3-7 kp. The amount of Eudragit used in the process can be easily varied more 50% on either side by adding/deleting generally acceptable pharmaceutical ingredients described earlier.

Capsule Filling

The 4 different tablets (steps 1,2 3 & 4) were filled into capsules by hand. The amount is shown below:

1 unit immediate release tablet-containing 150 mg Propafenone HCl

1 unit Matrix tablet containing 150 mg Propafenone HCl

1 unit delayed Matrix tablet containing 150 mg Propafenone HCl

1 unit prolonged Matrix tablet containing 150 mg Propafenone HCl

### EXAMPLE 3

Composition made in the form of Capsules Containing Multiple Units.

The Propafenone controlled release product is prepared by manufacturing one type of tablets, which afterwards is coated with 3 different types of film coating. The capsule ends up with 3 different types of tablets (one non-coated tablet, one EC-coated tablet, and prolonged EC-coated tablets).

Core Tablet Preparation-I

Propafenone hydrochloride 150 mg

Croscarmellose sodium 10 mg

Methocel E6 LV powder 3.8 mg

Purified water 34.2 mg (Methocel is dissolved in Purified water)

Additional purified water—15 mg

Magnesium Stearate 1.2 mg

The active drug Propafenone and Croscarmellose sodium is wetted with a Methocel/purified water solution and granulation is performed at an appropriate time & mixing intensity. The additional purified water may be added to achieve the desired granulation. The drying of the wet granulate is carried out in a Glatt fluid bed. The granules are dried to LOD of not more than 2 wt. %. The dried granulate is passed through a suitable sieve (#20 mesh). Magnesium Stearate is sieved through a #40 mesh and mixed with the sized granules in a V-Blender for 5 minutes. The tablet is compressed at 165-mg weight and hardness of 3-7 kp.

Core Tablet Preparation-II

Propafenone hydrochloride 300 mg

Croscarmellose sodium 20 mg

Methocel E6 LV powder 7.6 mg

Purified water 68.4 mg (Methocel is dissolved in Purified water)

Additional purified water—30 mg

Magnesium Stearate 2.4 mg

The active drug Propafenone and Croscarmellose sodium is wetted with a Methocel/purified water solution and granulation is performed at an appropriate time & mixing intensity. The additional purified water may be added to achieve the desired granulation. The drying of the wet granulate is carried out in a Glatt fluid bed. The granules are dried to LOD of not more than 2 wt. %. The dried granulate is passed through a suitable sieve (#20 mesh). Magnesium Stearate is sieved through a #40 mesh and mixed with the sized granules in a V-Blender for 5 minutes. The tablet is compressed at 330-mg weight and hardness of 4-10 kp.

Step 1—Non Coated Tablets

One batch of above Core tablets is not coated because it is used as an immediate-release unit. The tablet is a part of the content in the capsule.

Step 2—EC Coated Tablet

One batch of Core tablets-II is coated with an outer coat in a conventional perforated coating pan with a spray nozzle 1.5-mm and a spray pressure of 2.5 bar. The composition for the coating is shown below:

Pan Load 500 g

Eudragit L30 D 55 dispersion 260 g

Triethyl Citrate 8 g

Talc 20 g

Purified water 183 g

10 wt. % of dry matter was applied, calculated in the percentage of the core weight. It is possible to easily vary the coating level by adding certain phannaceutical excipients from 1 to 20 wt. %. Throughout the coating process the bed temperature was maintained in the interval from 30-38° C. by adjustment of the liquid inflow rate of the inlet temperature. The inlet air temperature was kept at approximately 45-55° C.

Step 3—Prolonged EC Coated Tablet

One batch of core tablets-I is coated with an outer coat in a conventional perforated coating pan with a spray nozzle 1.5-mm and a spray pressure of 2.5 bar. The composition for the coating is shown below:

Pan Load 500 g

Eudragit FS 30 D dispersion 260 g

Triethyl Citrate—4 g

Talc—39 g

Purified water—234 g

10 wt. % of dry matter was applied, calculated in the percentage of the core weight. It is possible to easily vary the coating level by adding certain pharmaceutical excipients from 1 to 20 wt. %. Throughout the coating process the bed temperature was maintained in the interval from 30-38° C. by adjustment of the liquid inflow rate of the inlet temperature. The inlet air temperature was kept at approximately 45-55° C.

Capsule Filling

The 3 different tablets (steps 1,2 & 3) were filled into capsules by hand. The amount is shown below:

1 unit Non-coated tablet containing 150 mg Propafenone HCl

1 unit EC-coated tablet containing 300 mg Propafenone HCl

1 unit prolonged EC-coated tablet containing 150 mg Propafenone HCl

### EXAMPLE 4

Composition Made in the Form of Capsules Containing Multiple Units.

The Propafenone controlled release product is prepared by manufacturing 3 types of tablets. The capsule ends up with 3 different types of tablets (one non-coated tablet, compressed & coated tablet having shorter lag-time and one compressed & coated tablet having longer lag-time tablets). The different between Example 1, 2 & 3 is the method producing types of tablets, which makes them time-specific rather than pH dependent.

Step 1—Non Coated Tablets

Core Tablet Preparation:

Propafenone hydrochloride 250 mg

Croscarmellose sodium 16.6 mg

Methocel E6 LV powder 6.3 mg

Purified water 56.8 mg (Methocel is dissolved in Purified water)

Additional purified water—20.3 mg

Magnesium Stearate 2 mg

The active drug Propafenone and Croscarmellose sodium is wetted with a Methocel/purified water solution and granulation is performed at an appropriate time & mixing intensity. The additional purified water may be added to achieve the desired granulation. The drying of the wet granulate is carried out in a Glatt fluid bed. The granules are dried to LOD of not more than 2%. The dried granulate is passed through a suitable sieve (#20 mesh). Magnesium Stearate is sieved through a #40 mesh and mixed with the sized granules in a V-Blender for 5 minutes. The tablet is compressed at 274.9-mg weight and hardness of 4-10 kp. The tablet is a part of the content in the capsule.

Step 2—Compressed & Coated Tablet Having Shorter Lag-Time

The composition of compressed tablets is shown below:

Propafenone hydrochloride 175 mg

Avicel PH 102 10.2 mg

Croscarmellose sodium 11.6 mg

Eudragit RL30D dispersion 151.2 mg

Eudragit RS 30D dispersion 16.8 mg

Triethyl Citrate 5 mg

Talc 20 mg

Purified water 142 mg

Magnesium Stearate 1.4 mg

The active drug Propafenone, Avicel PH 102 and Croscarmellose sodium is coated with Eudragit dispersion containing TEC & Talc diluted with water to solid content of about 23 wt. % using Glatt fluid bed coater/dryer. After about 175 mg of the coating suspension is sprayed, the coated powder is dried to LOD of not more than 2 wt. %. The dried coated powder is passed through a suitable sieve (#20 mesh). Magnesium Stearate is sieved through a #40 mesh and mixed with the sized coated powder in a V-Blender for 5 minutes. The tablet is compressed at 237.6-mg weight and hardness of 3-9 kp.

Coating of the Compressed Tablet

Above compressed tablet is coated with an outer coat in a conventional perforated coating pan with a spray nozzle 1.5-mm and a spray pressure of 2.5 bar. The composition for the coating is shown below:

Pan Load 500 g

Eudragit RS 30 D dispersion 260 g

Triethyl Citrate—8 g

Talc—39 g

Purified water—230 g

2 wt. % of dry matter was applied, calculated in the percentage of the core weight. It is possible to easily vary the coating level by adding certain pharmaceutical excipients from 1 to 20 wt. %. Throughout the coating process the bed temperature was maintained in the interval from 30-38° C. by adjustment of the liquid inflow rate of the inlet temperature. The inlet air temperature was kept at approximately 45-55° C. Optionally, surfactants such as sodium lauryl sulfate or Tween 80 may be used to enhance the drug release from the coating.

Step 3—Compressed & Coated Tablet Having Longer Lag-Time

The composition of compressed tablets is shown below:

Propafenone hydrochloride 175 mg

Avicel PH 102 10.2 mg

Croscarmellose sodium 11.6 mg

Eudragit RL30D dispersion 151.2 mg

Eudragit RS 30D dispersion 16.8 mg

Triethyl Citrate 5 mg

Talc 20 mg

Purified water 142 mg

Magnesium Stearate 1.4 mg

The active drug Propafenone, Avicel PH 102 and Croscarmellose sodium is coated with Budragit dispersion containing triethyl citrate & Talc diluted with water to solid content of about 23 wt. % using Glatt fluid bed coater/dryer. After about 175 mg of the coating suspension is sprayed, the coated powder is dried to LOD of not more than 2 wt. %. The dried coated powder is passed through a suitable sieve (#20 mesh). Magnesium Stearate is sieved through a #40 mesh and mixed with the sized coated powder in a V-Blender for 5 minutes. The tablet is compressed at 237.6-mg weight and hardness of 3-9 kp.

Coating of the Compressed Tablet

Above compressed tablet is coated with an outer coat in a conventional perforated coating pan with a spray nozzle 1.5-mm and a spray pressure of 2.5 bar. The composition for the coating is shown below:

Pan Load 500 g

Eudragit RS 30 D dispersion 260 g

Triethyl Citrate—8 g

Talc—39 g

Purified water—230 g

4 wt. % of dry matter was applied, calculated in the percentage of the core weight. It is possible to easily vary the coating level by adding certain pharmaceutical excipients from 1 to 20 wt. %. Throughout the coating process the bed temperature was maintained in the interval from 30-38° C. by adjustment of the liquid inflow rate of the inlet temperature. The inlet air temperature was kept at approximately 45-55° C.

Capsule Filling

The 3 different tablets (steps 1,2 & 3) were filled into capsules by hand. The amount is shown below:

1 unit immediate release tablet-containing 250 mg Propafenone HCl

1 unit compressed & coated tablets having shorter lag-time containing 175 mg Propafenone HCl

1 unit compressed & coated tablets having longer lag-time containing 175 mg Propafenone HCl

### EXAMPLE 5

Composition Made in the Form of Capsules Containing Multiple Units.

The Propafenone controlled release product is prepared by manufacturing 2 types of pellets. The capsule ends up with 2 different types of pellets (one non-coated pellet and one multicoated pellet)

Step 1—Non Coated Active Pellets

Active drug pellets is prepared in steps as below:

Active pellets-I preparation (First layer of the active):

Sugar spheres 45/60-mesh 280 mg

Propafenone hydrochloride, micronized 600 mg

Methocel E6 LV powder 60 mg

Purified water 2640 mg

Antifoam 30% dispersion—5 mg

Active pellets-II preparation:

Active pellets-I 440 mg

Propafenone hydrochloride 440 mg

Methocel E6 LV powder 40 mg

Purified water 1920 mg

Antifoam 30% dispersion—5 mg

Methocel solution is prepared by mixing purified water with Methocel until the solution becomes clear. The active drug Propafenone is then suspended into Methocel solution while mixing. After uniform suspension is achieved, add the antifoam suspension and continue mixing till desired suspension is achieved. The drug suspension is homogenized for not less than 5 minutes to achieve the desired suspension. This drug suspension is then sprayed onto sugar spheres using fluid bed coater (bottom spray technique) to achieve the active pellets. Optionally, the active pellets can be coated with a seal coat consisting of Methocel solution.

Step 2—Multicoated Pellets

The composition of mnulticoated pellets is shown below:

Inner Coat consisting of following Composition:

Non Coated Active pellets-I from Step 1 (First Layer of active) 500 g

Eudragit FS 30 D dispersion 260 g

Triethyl Citrate—4 g

Talc—39 g

Purified water—234 g

About 217 grams of Eudragit FS30D dispersion containing triethyl citrate & Talc was sprayed onto Active pellets-I using a fluid bed coater. It is possible to easily vary the coating level by adding certain pharmaceutical excipients from 1 to 20 wt. %. Throughout the coating process the bed temperature was maintained in the interval from 30-38° C. by adjustment of the liquid inflow rate of the inlet temperature. The inlet air temperature was kept at approximately 45-55° C.

Second layer of Active consisting of following composition:

Inner coated pellets from Step 2 500 g

Propafenone hydrochloride 490 mg

Methocel E6 LV powder 49 mg

Purified water 2156 mg

The entire drug suspension is sprayed in to Inner coated pellets using the same process and technique employed in the first step.

Outer coat consisting of following composition:

Second layer active coated pellets 1000 mg

Eudragit L30 D 55 dispersion 260 g

Triethyl Citrate 8 g

Talc 20 g

Purified water 183 g

266 grams of above suspension was sprayed on to second layer coated pellets It is possible to easily vary the coating level by adding certain pharmaceutical excipients from 1 to 20 wt. %. Throughout the coating process the bed temperature was maintained in the interval from 30-38° C. by adjustment of the liquid inflow rate of the inlet temperature. The inlet air temperature was kept at approximately 45-55° C.

Optionally: the seal coating solution consisting of Methocel or other polymer may be employed to make the coating process more efficient and/or to separate different coat from each other.

Capsule Filling

The 2 different pellets (steps 1 & 2) were filled into capsules by hand. The amount is shown below:

1 unit Non-coated pellets-II containing 200 mg of Propafenone HCl

1 unit of multicoated pellets containing 400 mg of Propafenone HCl

Dissolution Test:

A dissolution test was conducted on the novel compositions as specified in the above examples utilizing current USP Test for Apparatus II (paddles) at rotating at 50 r.p.m. A sample is dissolved in an aqueous medium previously degassed and equilibrated to 37° C. The media are pH 2.0, pH 5.5, pH 6.8 & pH 7.5 phosphate buffer for all the examples except for example 4 where deionized water was used. A sample of unknown volume is withdrawn at designated time intervals from the bath as directed and subjected to a suitable assay procedure. The mg released of Propafenone hydrochloride as a function of time is tabulated as the dissolution profile.

### EXAMPLE 1

### Dissolution Profile

Non coated tablets—test was conducted in pH 2 for 1 hour. Two samples were withdrawn, 30 minutes & 1 hour. About 80-90 wt. % of the dose of Propafenone HCl was released in the first 30 minutes.

EC Coated tablets—test was conducted in pH 2 for 2 hour followed by pH 5.5 for 1 hour. Two samples were withdrawn in each media. 1 hour & 2 hour in pH 2.0 and 30 minutes & 1 hour in pH 5.5. After 2 hours in pH 2.0 less than 5 wt. % release of Propafenone HCl was seen. About 80-90 wt. % of the dose of Propafenone HCl was released in the first 30 minutes in pH 5.5.

Delayed EC Coated tablets—test was conducted in pH 2 for 2 hour followed by pH 6.8 for 1 hour. Two samples were withdrawn in each media. 1 hour & 2 hour in pH 2.0 and 30 minutes & 1 hour in pH 6.8. After 2 hours in pH 2.0 less than 5 wt. % of the dose of Propafenone HCl was released. About 80-90 wt. % of the dose of Propafenone HCl was released in the first 30 minutes in pH 6.8.

Prolonged EC Coated tablets—test was conducted in pH 2 for 2 hour followed by pH 5.5 for 2 hours followed pH 7.5 for 1 hour, Two samples were withdrawn in each media. 1 hour & 2 hour in pH 2.0 and pH 5.5 and 30 minutes & 1 hour in pH 7.5. After 2 hours in pH 2.0 and pH 5.5 less than 10 wt. % of the dose of Propafenone HCl was released. About 80-90 wt. % of the dose of Propafenone HCl was released in the first 30 minutes in pH 7.5.

Example 2 & 3 are exhibited similar profile as described in Example 1. They were targeted to provide the similar dissolution profile. The only difference is that they are formulated differently.

### EXAMPLE 4

### Dissolution Profile

Non coated tablets—test was conducted in distilled water for 1 hour. Two samples were withdrawn, 30 minutes & 1 hour. About 80-90 wt. % of the dose of Propafenone HCl was released in the first 30 minutes.

Compressed & Coated tablet having shorter lag-time—test was conducted in distilled water for 4 hour. Three samples were withdrawn; 30 minutes, 2 hour & 4 hour. After 30 minutes and 2 hours less than 10 wt. % of the total dose was released. About 80-90 wt. % of the total dose of Propafenone HCl was released in the fourth hour.

Compressed & Coated tablet having longer lag-time—test was conducted in distilled water for 8 hour. A total of five samples were withdrawn; 30 minutes, 2 hour, 4 hour, 6 hour & 8 hour. After 30 minutes and 2 hours less than 10 wt. % of the total dose of Propafenone HCl was released. After 6 hour about 10 wt. % of the total dose is released. Remaining About 80 wt. % of the total dose of Propafenone HCl was released in the eight hour.

### EXAMPLE 5

### Dissolution Profile

Non coated pellets—test was conducted in pH 2.0 for 1 hour. Two samples were withdrawn, 30 minutes & 1 hour. About 80-90 wt. % of the total dose of Propafenone HCl was released in the first 30 minutes.

Multicoated pellets—test was conducted in pH 2 for 2 hour followed by pH 5.5 for 2 hours followed pH 7.5 for 1 hour. Two samples were withdrawn in each media. 1 hour & 2 hour in pH 2.0 and pH 5.5 and 30 minutes & 1 hour in pH 7.5. After 2 hours in pH 2.0 less than 10 wt. % of the total dose present in the second drug layer was released. After 2 hours in pH 5.5 about 80-90 wt. % of the total dose of Propafenone HCl present in the second drug layer was released. About 80-90 wt. % of the dose present in the first drug layer was released in the first 30 minutes in pH 7.5.

What is claimed is:

1) A single drug delivery system dosage for a drug metabolized by the liver of humans or animals, comprising an oral dosage of said drug having at least one part which releases immediate followed by a delayed release, however said delayed release, is immediate upon release.

2) The delivery system of claim 1, wherein said drug is an antiarrlhythmic.

3) The delivery system of claim 2, wherein said drug is propafenone HCL.

4) The delivery system of claim 1, wherein said delayed release is at least one hour after said immediate release.

5) A single drug delivery system for multiple dosages of drug in ones gastrointestinal tract, comprising an oral dosage of said drug having an initial portion that releases said drug immediately in said gastrointestinal tract, and a delayed release portion that releases drug upon a time delay, but once released, all of said delayed release portion of said drug is immediately released.

6) The drug delivery system of claim 5, wherein said drug is a cardiac arrhythmia drug.

7) The drug delivery system of claim 6, wherein said drug is propafenone.

8) The drug delivery system of claim 5, wherein said delayed released portion is at least one hour after said initial portion of said drug.

9) The delivery system of claim 5, wherein said drug is sufficient for once a day dosing.

10) The delivery system of claim 5, wherein said delayed release portion is coated with a material that delays its absorption in said gastrointestinal tract.

11) A single drug delivery system for releasing multiple dosages of drug in ones gastrointestinal tract, comprising an oral dosage of said drug having an initial portion that releases said drug immediately in said gastrointestinal tract, a second portion that is delayed released after said initial release, and a third portion that is delayed released after said second portion, each of said second and said third portion, once released, is completely and immediately released in said gastrointestinal tract.

12) The single drug delivery system of claim 11, wherein said second portion is released at least an hour after said initial portion, and said third portion is released at least one hour after said second portion.

13) The single drug delivery system of claim 11, wherein said drug is a sodium channel blocker, a beta blocker, a potassium channel blocker, a calcium channel blocker, or a mixture of two or more of the blockers.

14) The single drug delivery system of claim 13, wherein said drug is propafenone HCL.

15) The single drug delivery system of claim 11, wherein said second portion is coated with a material that absorbs slowly in said gastrointestinal tract, and said third portion is coated with a thicker coating of said material.

16) The single drug delivery system of claim 11, wherein said second portion is coated with a first material that absorbs slowly in said gastrointestinal tract, and said third portion is coated with a second material that absorbs more slowly than said first material.

17) The single delivery system of claim 15, wherein said material is cellulose or polymer based.

18) The single delivery system of claim 17, wherein said polymer is acrylic.
